(19) United States
(12) Patent Application Publication
(10) Pub. No.: US 2020/0321131 A1
(43) Pub. Date: Oct. 8, 2020

(54) Method of Minimizing Patient Risk

(71) Applicant: Anand Mansukh Pankhania, Berkshire (GB)

(72) Inventor: Anand Mansukh Pankhania, Berkshire (GB)

(21) Appl. No.: 16/843,030

(22) Filed: Apr. 8, 2020

(30) Foreign Application Priority Data
Apr. 8, 2019 (GB) 1904949.3

(51) Int. Cl. G16H 50/30; G16H 40/20; G16H 50/70; G16H 10/60; G16H 20/10; G16H 70/40; G16H 15/00

(52) U.S. Cl. CPC G16H 50/30; G16H 40/20; G16H 50/70; A61B 5/7275; G16H 20/10; G16H 70/40; G16H 15/00; G16H 10/60

(57) ABSTRACT

A method of data stratification is provided for management of a patient group according to individual risk factors. The method comprises the step of: a) receiving a patient data, wherein the patient data comprising: i. a unique identifier; ii. a first medication list comprising currently prescribed medications. The method further comprises the steps of: b) producing a drug data by comparing the first medication list with a drug risk classification database; c) determining a new risk value using the drug data and the patient data; d) inserting the patient data into a memory; and e) accessing the memory and ordering the unique identifiers into ordered unique identifiers, wherein the ordered unique identifiers being ordered according to the corresponding new risk values. The invention may in some embodiments generate new risk values according to a supervised machine learning model, which may include a neural network, and implemented using a machine learning module. The present method preferably provides improved management of a patient group wherein a number of potentially interacting risk factors are accommodated.

### CROSS-REFERENCE TO RELATED APPLICATIONS

This Application claims priority under the Paris Convention to United Kingdom Patent Application No. 1904949.3, filed Apr. 8, 2019, the entire content of which is herein incorporated.

### BACKGROUND OF THE INVENTION

### 1. Field of Invention

The present invention relates to methods of data stratification for risk management, and particularly to data stratification for managing risks associated with prescribed medications.

### 2. Description of the Related Art

When admitted to a hospital, a patient may be prescribed one or more medications, and said patient may have multiple complex co-morbidities. The prescribed medications therefore require regular assessments to ensure that they are prescribed appropriately and accurately for a patient. Accurate prescribing can depend on many clinical factors. Medications present significant financial and clinical risks to any organization, for example due to missed doses; inaccurate medicine reconciliation; medications not being prescribed accurately for a specific clinical picture; all of which can lead to sub-optimal therapy. Such adverse effects can range from mild to severe depending upon the particular medication or medications prescribed, and can either cause harm to the patient, or reduce patient adherence to a recommended dosage regimen. An increased number of medications increases the likelihood of drug-drug interactions, which can have large effects on the efficacy of one or more drugs. This is particularly relevant to high-risk medications such as insulins; or narrow therapeutic drugs such as vancomycin; or phenytoin; all of which require specific drug monitoring.

A high-risk patient is defined as a patient who's required specialist input is determined by the types of drugs they are prescribed for a specific disease state such as Parkinson's disease or diabetes. Such patients are typically prescribed more complex drug regimens which have been shown to have a higher drug prescription discrepancy rate. A high-risk patient could be prescribed a drug which requires careful drug monitoring and dose adjustments based on the patient's blood results indicating, for example, renal function.

Management of individual medication plans is typically carried out by a doctor and a pharmacy team including a pharmacist. Currently, pharmacy teams typically only use a rudimentary software implementation to facilitate a number of their daily tasks. Such tasks may, for example, include determining if medications are correctly presented on a prescribing chart, determining whether medications require dose adjustment according to a patient's clinical observations, and determining an optimum dosage regimen for an individual.

There are currently an inadequate number of resources to allow a pharmacist to perform these tasks in a safe, effective, timely and equitable manner and as a result, patients experience unintended medication discrepancies which can severely impact upon the patient's length of stay and key outcome data, such as mortality and morbidity. Other potentially hazardous consequences may, for example, include medications being missed off prescribing charts; drug history reviews being too infrequent or missed entirely; and high risk patients not being suitably represented in an appointment ordering system.

Individual scoring systems are currently available for a number of different risk factors, such as, for example, the CHADS-VASC score for atrial fibrillation, and can give an outcome score for a patient based on individual risk factors. Such scoring systems are isolated however, being related to an individual risk factor. Indications of high risk individuals being admitted to hospital are presently used, which usually only indicates number of admissions, and does not provide any information to permit risk-based stratification of data or the ability to learn about risks based on the data presented.

To the applicant's knowledge, no system presently exists which provides an outlook on multiple, potentially compounding or otherwise interacting, risk factors for use in managing a patient group.

It is therefore desirable to provide a method of data stratification for management of a patient group according to multiple interacting risk factors.

### BRIEF SUMMARY OF THE INVENTION

In accordance with a first aspect of the present invention, there is provided a method of data stratification for management of a patient group according to individual risk factors, the method comprising the steps of:
a) by one or more computing devices, receiving a patient data, the patient data comprising:
   i. a unique identifier; and
   ii. a first medication list comprising currently prescribed medications;
b) by one or more computing devices, producing a drug data by comparing the first medication list with a drug risk classification database;
c) by one or more computing devices, determining a new risk value using the drug data and the patient data;
d) by one or more computing devices, storing the patient data, and optionally the drug data, into a memory; and
e) by one or more computing devices, accessing the memory and ordering the unique identifiers into ordered unique identifiers, the ordered unique identifiers being ordered according to the corresponding current risk values.

In the context of the present invention, the terms “medication” and “drugs” will be understood by the skilled addressee to mean substantially the same thing, and these terms are used interchangeably herein.

In preferable embodiments, the memory comprises a transient memory, such as random access memory, and therefore the patient data and/or drug data and/or risk value may comprise transient data. In such embodiments, the determination of risk values and ordering of unique identifiers is a dynamic process and does not rely on a hard-stored database of patient data.

Embodiments will be appreciated wherein the memory is a physical memory, such as a hard drive. In such embodiments, the physical memory may comprise a patient database having patient data contained in database entries according to each corresponding unique identifier. In such embodiments, step d) may comprise, by the one or more computing devices, storing the patient data, and optionally the drug data, into an entry of the patient database according to the unique identifier. In such embodiments, step e) may comprise, by the one or more computing devices, accessing the patient database and ordering the unique identifiers into ordered unique identifiers, the ordered unique identifiers being ordered according to the corresponding current risk values.

Steps of the present method are preferably performed by a processor of the one or more computing devices. Said processor may, in some embodiments, make use of a machine learning module of the one or more computing devices. For example, during step c), the determination of a new risk value may be performed by the one or more computing devices using the machine learning module of the one or more computing devices. In such embodiments, the machine learning module may make use of a supervised learning environment for a neural network.

In such examples, the one or more computing devices may be arranged to generate the new risk value, for a given individual, based on all input data (the drug data and the patient data), which may include the adding of weights, biases and/or activation factors depending on the particular input data. The neural network may, for example, be built for a .net framework for use by the one or more computing devices in implementing the present method.

Step c) may, in some embodiments, comprise generating, by the one or more computing devices (which may utilise the machine learning module), a model which may use the input data (the drug data and the patient data) to generate the new risk value (characterising a prediction of the risk for the particular individual patient). In some embodiments, said generating comprises, by the one or more computing devices, specifying a linear algorithm, which then produces a model which combines input data with a set of weights, biases and/or activation factors to calculate the new risk value. Such implementations of said generating in step c) work well for linearly separable features and the weights, biases and activation factors are preferably estimated during training of the linear algorithm, which is then applied to a regression binary trainer.

The new risk value for the given individual is preferably generated, in step c), by predicting targets using a linear binary classification model that is trained by importing training data (which may include training data for all expected input data—patient data and drug data). The generating in step c) preferably uses a gradient descent that makes predictions by finding a separating hyperplane, with the prediction given by determining which side of the hyperplane a particular input data falls into. Said generating in step c) preferably uses a weighted sum where weights, biases and activation factors are preferably computed during said generating.

Individual risk factors are used in the present invention to determine corresponding risk values according to the severity of each said risk factor. The risk values are then used cumulatively to determine a risk presented by an individual patient and which can then be used to, for example, order said patients according to their respective risk presented, such that patient ordering for appointments and ward visits is simplified.

The present method preferably sets risks numbers based on critical factors and orders them into a list with live updates continuously. Once, for example, a pharmacist has seen a patient, the risk value for said patient may set to 0 until something has changed, which may be after the pharmacist has seen the patient. A system implementing the present method may also allow automatic procurement/dispensing/purchasing of medications for a patient once a medication has been verified/screened by an authorised personnel, such as a pharmacist. The system may calculate the number of days needed for a prescribed course of medication, or a required medication duration, such as a 14-day treatment period, and then procure the corresponding quantity of the required medication. The system may reorder the course again in, for example, 13 days, to ensure a continuous supply of the required medication.

If a drug is verified, the medication upon ordering preferably goes straight to being dispensed, otherwise the medication requires authorization from an authorized personnel, prior to being dispensed.

A pharmacist typically has 30 minutes to 7 hours to see a varying number of patients to ensure they are reviewed, and that their prescribed medication is safe and optimized. It is difficult to review all patients in a day, which means that if patients are seen in a suboptimal order, the most at-risk patients may potentially be missed at a critical moment. The present system preferably risk-stratifies a patient group in a systematic order based on key risk factors to ensure that the most at-risk patients are seen first. The present system preferably provides live, real-time updated listing, therefore providing a medical professional with live updates on whomever presents the next highest risk, such that the most at-risk patients remain at the top of the list. Such generation and updating may be performed by, or with the aid of, a machine learning module. The present method therefore preferably provides for safe prescribing of patient as a priority.

A system utilizing the present method may preferably accept live updates from a medical professional during visitations or appointments and may preferably be integrated within procurement and purchasing systems to permit automatic procurement of medications based on what has been screened by a pharmacist or medical professional.

The present method therefore preferably provides advantages over current methods including improved safety, efficiency and time saving. Systems utilizing the present method may preferably include communication with one or more remote data sources (such as for the drug risk classification database) and collates information for easy interpretation. In an example embodiment, such systems may source patient data, drug data, clinical observation data or other medical record related data from the remote data sources and parses and formats said data to create a user-friendly presentation of said data. The present invention also preferably allows communication between different healthcare professionals. For example, any interactions with a system implementing the present method, such as, for example, the ticking of a box indicating that an individual appearing on the unique identifier list has been seen by a healthcare personnel, will appear as ticked to all other users.

In some preferable embodiments, when the next individual arrives (which may be as a patient on a ward), the present method preferably further comprises, by the one or more computing devices, predicting a total risk of said patient, generated as a new risk value. Said risk may be based on various weighting and individual accumulated risk factors, and may include the use of machine learning module, to inform the reordering of the ordered unique identifiers.

Additional embodiments preferably include the step of, by the one or more computing devices: generating a prediction of a drug in the first medication list, or a group of drugs in the first medication list, which are contributing to an individual/patient's symptoms or condition (included within the patient data). Such a step may be incorporated into step c) and may utilize the patient data and the drug data, preferably in combination with a machine learning module, to generate a new risk value accordingly. Such a step which, coupled with step e) of ordering the unique identifiers, is preferably beneficial for symptom or condition prevention, alleviation or treatment. Such preferable embodiments preferably permit prioritizing the individual/patient to be seen and also advises what needs to be reviewed by an appropriate personnel.

In some embodiments, if an individual/patient symptoms or condition is continuously improved/reduced (which may be determined according to the patient data and/or drug data), the method may additionally comprise the step of, by the one or more computing devices, analyzing the patient data and/or the drug data and analyzing a drug in the first medication list which poses a risk. Such a step may be incorporated into step c) and informs the generating of the new risk value. Such an effect on risk value generation, coupled with the ordering in step e), preferably highlights predictions of possible future issues to relevant personnel for review at an appropriate priority level. The present method may use a machine learning module to continue to learn and identify trends in successive patient data, drug data and/or new risk values to improve efficiency of the present method.

For example, if a patient has, according to historic patient data/drug data/new risk values/ordered unique identifiers, been consistently positioned first (or close to first) in the ordered unique identifiers whose unique identifier actually subsequently appears further down the list of ordered unique identifiers, the present method may (preferably with the aid of a machine learning module) determine the historical trend, to inform generating of the new risk value either presently or at a future instance. Such conflicts with historical trends may be used in an additional step of the method to provide additional supervised learning to the machine learning module. Such anomalies may therefore not be overlooked or understated and can be effectively managed.

The present method preferably can be used in conjunction with a system providing directions and quantities for ordering through an automated labelling and dispensing process of a pharmacy, and provide data ready for checking by a professional or automated checking process. The data, such as the ordered unique identifiers, is preferably arranged to be paperless and visualized on display screens to allow for medication to be assembled safely and accurately and then given to a patient.

Preferably the drug risk classification database comprises one or more drug entries comprising a drug identifier and a corresponding drug risk value. Embodiments will be appreciated wherein the drug risk database does not comprise drug risk values, and instead comprises drug classification data. Said drug classification data may be used in the present method, for example by the one or more computing devices in step c), to allocate a drug risk value to a drug in real time during the implementation of said method. Examples of drug risk classifiers may, for example, include drug category/schedule; known or theorized drug toxicity characteristics (such as, for example, nephrotoxicity or hepatotoxicity); missed dose risk; time critical drug; effect of missed dosage; blood/electrolyte/other monitoring required; daily monitoring required; drug-drug interactions; known off-target effects; detriments if prescribed incorrectly. Embodiments will be appreciated wherein the drug risk classifiers are accompanied by a corresponding risk value, and may be used to allocate an overall drug risk value to the drug, such as, for example, by summing said corresponding risk values.

Preferably the patient data further comprises:
iii. a current risk value.

In embodiments wherein the patient data comprise a current risk value, the patient data may have been stored in, and accessed from, a physical memory, which may comprise a patient database having database entries according to each unique identifier. Said current risk value may therefore have been stored in said physical memory by the one or more computing devices during step d) of a previous iteration of the present method. In such embodiments, step c) of the present example iteration may comprise, by the one or more computing devices, changing the current risk value to the new risk value. In said present example iteration, step d) of the iteration may either comprise, by the one or more computing devices, storing said patient data in a transient memory or storing said patient data in the patient database, in a database entry according to the unique identifier of the patient data. In such embodiments, in the ordering step e), the patient database may be accessed and the unique identifiers of the database entries therein may be ordered by the one or more computing devices according to corresponding risk values. Thereby, the present method may either provide a dynamic use of transient memory, or a more long-term use of physical memory in assigning risk values to unique identifiers.

Preferably, the patient data further comprises:
iv. a period since a previous appointment.

In embodiments wherein the patient data comprises a period since a previous appointment, the period since a previous appointment (which may in some embodiments comprise a drug history review) may be used to calculate the new risk value. An extended period since a previous appointment may present a greater risk to patient, particularly if the period represents a period since a previous drug history review. The term “previous” will be understood to refer also to a patient who has not received a first appointment. In such cases the period will be understood to refer to the period since admission to a hospital or since registration with patient database, at a doctor's surgery for example.

Preferably the method further comprises the step of:
g) by one or more computing devices, generating a report of the ordered unique identifiers.

Preferably step d) comprises storing the drug data into a patient database entry according to the unique identifier and step e) further comprises, by the one or more computing devices:
identifying a plurality of unique identifiers having the same corresponding current risk value;
comparing the drug data corresponding to the plurality of unique identifiers to calculate an order of the plurality of unique identifiers; and
ordering the plurality of unique identifiers.

Individual patients may, according to the risk classification of steps a) to d), present the same risk value. Such nested ordering is preferably necessary when more than one patient database entry comprises the same risk value, and therefore the same potential risk severity. In this case, the ordering of the database entries (represented by unique identifiers) is carried out within the ordered unique identifiers. This nested ordering may preferably occur immediately following, or subsequent to, the ordering of the unique identifiers into the ordered unique identifiers in step e).

Preferably step c) comprises, by the one or more computing devices:
determining that the period since a previous appointment is zero; and
determining the new risk value of zero.

The present method may comprise a step wherein following visitation with a patient, the period since a previous visitation is set to 0. In such a circumstance, the current risk value is preferably then set to 0. A timer may be used in a system utilizing the present method to maintain the current risk value at 0 for a predetermined time period, such as, for example, 24 hours. Therefore the present method may permit other at-risk patients to succeed more-recently seen, higher-risk patients in the ordered unique identifiers.

Each time, for example, a new ward is visited, the present method may be used to determine a suitable order of ward patients for a medical professional to visit according to the severity of their at-risk status. Risk values may then be updated accordingly, except for those patients seen earlier that day, the risk values for whom will preferably be maintained at 0. At a predetermined time (such as midnight for example), or after a predetermined time period (such as 24 hours for example), all current risk values may be recalculated as per the steps of the present method. In an example, a risk value of 0 may be assigned to a patient if seen by a pharmacist earlier in the day. Subsequently, if said patient data is prescribed additional (or different) medication later that day, patient data may be received for said patient having a different first medication list to when the patient was assigned a risk value of 0. At this point, later in the day, a new risk value is generated for the patient according to the new first medication list. In this example, if a pharmacist visits with every patient by 12 pm, and no further changes are made to the patient data, every patient will have a risk value of 0. At 4 pm, if a patient's first medication list has changed, new risk values will be generated for said patient. Upon the subsequent generation of a patient risk order, said patient will then appear higher, or at the top of, said order by risk number. In this example, all risk values are recalculated at midnight.

Preferably the patient data further comprises:
v. a period since a previous drug history review.

Preferably step c) comprises, by the one or more computing devices:
determining that the period since a previous drug history review is zero; and
determining the new risk value of zero.

Preferably the patient data further comprises:
vi. a second medication list comprising previously prescribed medications.

Preferably step b) comprises, by the one or more computing devices:
comparing the second medication list with the first medication list.

In some embodiments, the drug risk classification database may be local to the one or more computing devices. In other embodiments, the drug risk classification database may be external to the one or more computing devices and operated and maintained by a third party for example. In such embodiments the one or more computing devices access the external drug risk classification database and obtain external drug risk classification data for comparison with the first medication list in order to produce the drug data.

Preferably the patient data further comprises:
vii. a clinical observation data;

Preferably the clinical observation data comprises one or more selected from the group: an allergy classifier; a missed dose classifier; a renal competency classifier; a hepatic competency classifier; a blood marker classifier; a patient demographic classifier; an age classifier; a race classifier; a genetic marker classifier; a disease classifier; a comorbidity classifier. Other suitable examples of clinical observation data for use with the present invention will be appreciated, and may, for example, include a bone density classifier for chemotherapeutic drugs expected to affect bone density. Further examples of clinical observation data are included in Tables 10 to 12 below. Demographic classifiers, such as those for age, race and genetic markers may be useful for identifying potential risks for patients who are prescribed drugs known to have adverse side effects in specific population groups. For example, single nucleotide polymorphisms (SNPs) which occur more frequently in a specific population group may lead to a higher risk value being allocated to individuals from said population group, when prescribed medication known to have pharmacokinetic properties which are affected by said SNPs. Disease and comorbidity classifiers may be useful for identifying patients for whom medication typically prescribed for one condition may be contraindicated for another condition. In such circumstances, careful prescribing and dosing is required. An example might include a patient diagnosed with diabetes, stroke and chronic kidney disease, for whom diabetes and stroke drugs which are primarily cleared renally will need to be carefully considered.

Preferably the drug risk classification database comprises one or more drug entries contained in one or more drug category entries, wherein each drug entry comprises a corresponding drug risk value.

Preferably the drug category entries comprises one or more selected from the group:
i. unverified drugs;
ii. controlled drugs;
iii. non-formulary drugs;
iv. venous thromboembolism drugs;
v. drug-drug interactions.

Preferably the drug data comprises at least one drug category quantity, wherein the at least one drug category quantity corresponds to a number of the drug entries within said drug category which are present in the first medication list.

In accordance with a second aspect of the present invention, there is provided a computer program product including a program for a processing device, comprising software code portions for performing the method steps of any method according to the first aspect of the present invention when the program is run on the processing device.

Preferably the computer program product comprises a computer-readable medium on which the software code portions are stored, wherein the program is directly loadable into an internal memory of the processing device.

In accordance with a third aspect of the present invention, there is provided a risk stratification system arranged to perform the method steps of the first aspect of the present invention, the system comprising a processing device having a processor arranged to process a computer program product according to the second aspect of the present invention.

Other features and aspects may be apparent from the following detailed description, the drawings, and the claims.

### BRIEF DESCRIPTION OF THE SEVERAL VIEWS OF THE DRAWINGS

Specific embodiments will now be described by way of example only, and with reference to the accompanying drawings, in which:

FIG. 1 shows a flow chart of an example embodiment of a method according to the first aspect of the present invention;

FIG. 2 shows a schematic diagram of an example embodiment of a system according to the third aspect of the present invention arranged to process a computer program product of the second aspect of the present invention and perform a method of the first aspect;

FIG. 3 shows a flow chart of an example implementation of step c) of the method of FIG. 1.

### DETAILED DESCRIPTION OF THE INVENTION

Reference will now be made to the example embodiments of the present general inventive concept, examples of which are illustrated in the accompanying drawings and illustrations. The example embodiments are described herein in order to explain the present general inventive concept by referring to the figures.

The following detailed description is provided to assist the reader in gaining a comprehensive understanding of the structures and fabrication techniques described herein. Accordingly, various changes, modification, and equivalents of the structures and fabrication techniques described herein will be suggested to those of ordinary skill in the art. The progression of fabrication operations described are merely examples, however, and the sequence type of operations is not limited to that set forth herein and may be changed as is known in the art, with the exception of operations necessarily occurring in a certain order. Also, description of well-known functions and constructions may be omitted for increased clarity and conciseness.

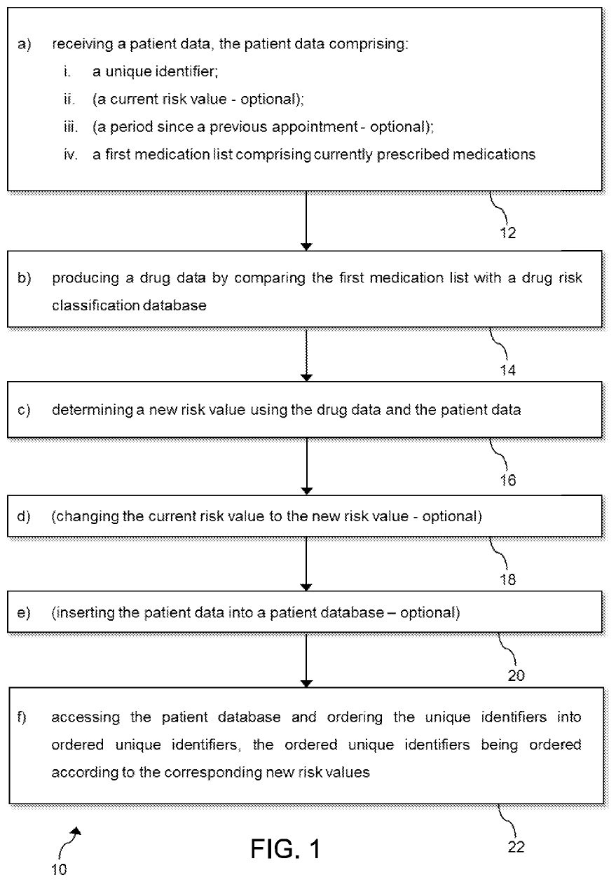

Referring to FIG. 1, an example method 10 according to the first aspect of the present invention is shown, the method comprising the steps of:
a) receiving a patient data 12, the patient data comprising:
   i. a unique identifier;
   ii. (a current risk value—optional);
   iii. (a period since a previous appointment—optional);
   iv. a first medication list comprising currently prescribed medications;
b) producing a drug data by comparing the first medication list with a drug risk classification database 14;
c) determining a new risk value using the drug data and the patient data 16;
d) (changing the current risk value to the new risk value—optional) 18;
e) inserting the patient data into a patient database 20; and
f) accessing the patient database and ordering the unique identifiers into ordered unique identifiers, the ordered unique identifiers being ordered according to the corresponding new risk values 22.

In accordance with the example method 10 shown in FIG. 1, a method is provided to carry out data stratification for risk management, the method being performable by a computer. In the example embodiment 10, a computer receives a patient data 12, the patient data comprising data associated with a particular patient having a unique identification (which in the case of the example shown, is the particular patient's name). An example of said patient data is shown below in Table 1, wherein each individual row represents a patient data.

TABLE 1
Patient Data

| | | Days since | | Clinical |
|---|---|---|---|---|
| Patient | Current Risk | most recent | Drugs presently | observations- |
| Identifier | Value | appointment | prescribed | disease |
| Patient 1 | 5 | 5 | Methadone | Opioid addiction |
| Patient 2 | 2 | 2 | Azithromycin | Sepsis |

The patient data in the example shown comprises a “current risk value”, the “current risk value” being a predetermined numerical data value defined by the cumulative sum of individual risk values attributed to corresponding individual “risk factors” recorded for a particular patient. The “risk factors” in the embodiment 10 shown comprise, for example, drugs presently prescribed; drugs previously prescribed; a period since a previous appointment; a period since a previous drug history review; and a number of clinical observations, which may include alerts or action points needed to be carried out from or to a medical practitioner, such as a pharmacist, doctor or nurse. The present example 10 shows an embodiment wherein Patients 1 and 2 have been previously admitted to a hospital and therefore their corresponding patient data comprises a “current risk value” and a “period since a previous appointment”. It would be understood that these aspects of the patient data in the example shown, together with that of step d) 18, are optional and provided for only in case wherein the present method is performed on patient data for patients already having a risk value and already admitted to hospital. This optional status is denoted by providing these aspects of the method inside parentheses as can be seen in FIG. 1. It would be understood that embodiments of the method performed using patient data of patients recently admitted to hospital may not comprise a “current risk value” or a “period since a previous appointment”, and therefore wherein step d) 18 is not performed. Other embodiments will be appreciated wherein said recently admitted patients may be allocated a high risk value such that said patients are seen as a priority.

The patient data further comprises a period since a previous appointment of the particular patient, represented by a numerical data value. In the case of the example shown the period since a last appointment is a measure of days. Embodiments will be appreciated wherein the period is a measure of weeks, or any other suitable time denomination. In the example embodiment shown, the period since a last appointment is a period since the most recent visit from a medical practitioner to the particular patient on a hospital ward. Embodiments will be appreciated wherein the period since a previous appointment is not the period since the most recent appointment, and may not be related to a visit on a ward, and may in some embodiments be a period since a previous local GP surgery appointment.

The patient data further comprises a first medication list, the first medication list comprising a text-based list of medications prescribed to the particular patient. Embodiments will be appreciated wherein the first medication list comprises a numerical-based list, wherein each numerical data therein is a key uniquely attributed to an entry in a medication table or database.

In the example shown, the patient data is retrieved by the computer from a digital medical history file of the particular patient located on a local hospital server. Embodiments will be appreciated wherein one or more individual components i-iv of the patient data is retrieved by the computer from one or more data sources, which may include local or national patient databases.

Following retrieval of the patient data by the computer 12, the method of the embodiment 10 shown comprises the step of producing a drug data by comparing the first medication list (of presently prescribed medication) with a drug risk classification database 14. The drug risk classification database comprises a list of drugs and their corresponding risk values, which in the example shown are represented in an associative array comprising drug identifiers (in the case shown, drug names) each linked to a corresponding risk value. An example structure of such an associative array is shown in Table 2, comprising drug names and their corresponding risk values (which may in turn be generated according to a supervised machine learning model and implemented using a machine learning module). Other suitable data structures will be appreciated by the skilled addressee, and may comprise, for example, a hash table, a dictionary, a tuple, or a database among others which will be appreciated.

TABLE 2
Individual drug risk values

| Drug Name | Risk Value |
|---|---|
| Methadone | 4 |
| Suboxone | 5 |
| Lithium | 4 |
| Clozapine | 5 |
| Azathioprine | 2 |
| Mycophenolate | 2 |
| Ciclosporin | 2 |
| Sirolimus | 5 |
| Tacrolimus | 5 |

Each particular drug is assigned to a drug risk category, or “Schedule” (such as, for example, the Controlled Drug (CD) Schedules), which in the example shown are listed from “Schedule 1” to “Schedule 5” in descending order of risk severity, each of which are allocated a corresponding risk value. Assignment of a particular drug to a category can preferably be altered by a medical practitioner having appropriate permissions. In the case of the CD Schedules, these are typically determined by local government and may not be alterable in the system. Such a reassignment may be due to recent regulatory changes relating to a particular drug, which may cause prescription of the drug to present either more or less of a concern to doctors and/or pharmacists. Alternatively, or in addition, embodiments will be appreciated wherein the drug risk categories are “high risk”, “medium risk”, and “caution risk”, wherein each category comprises a differing risk value determining the severity of risk posed by drugs of each category. Embodiments will be appreciated wherein risk values are assigned to individual drugs and not categories, and may be data-driven and may be adjusted in real-time according to data available via, for example, a third party API.

Each drug may be linked to a “screening status” which may either be “screened” or “unscreened”, wherein drugs in each of the “screened” and “unscreened” categories is allocated a corresponding risk value, wherein the risk value for “screened” drugs may be 0, and the risk value for “unscreened” drugs may be greater than zero, and may optionally be subject to a number of unscreened drugs present in the first medication list. Alternatively, the drugs may be allocated a “verification status”—either “verified” (which may be equivalent to “screened”) or “unverified (UV—which may be equivalent to “unscreened”)”—wherein like the “screened” and “unscreened” statuses, the drugs in each category are allocated a risk value according to the corresponding risk posed. Assignment of a particular drug to a status can preferably be altered by a medical practitioner having appropriate permissions, and may be in response to the appropriate medical practitioner confirming that such a drug is suitable for the patient. Until such an approval or confirmation from the appropriate medical practitioner, it may be the case that the particular drug is not sourced for the patient. Screening/verification status is typically linked to an individual drug, with each drug being screened independently and separately. Alternative embodiments will be appreciated wherein the “screening status” or “verification status” may not be linked to individual drugs, but to a whole first medication list, or to one or more groups of medications within the first medication list. In such embodiments, an authorised professional may review an entire first medication list, or one or more specific groups requiring approval, and may alter the “verification status” or “screening status” from “unverified” to “verified” or “unscreened” to “screened”. Unscreened or unverified drugs have not been checked by an authorised professional and therefore carry a risk of being inaccurately prescribed or dosed.

Statuses such as “screened” or “unscreened” and “verified” and “unverified” may be utilised by a purchasing or procurement system utilising or integrating with the present method or a system performing the present method, arranged to automatically purchase or procure medications either from a pharmacy department in a larger medical facility or directly from a supplier. Medications may, for example, only be automatically purchased or procured by said system if said drugs are “screened” or “verified” by appropriate personnel. Procurement may involve, for example, confirming that a medication may be dispensed from a pharmacy or pharmacy department. Said dispensing can therefore occur automatically without the need to have input from a pharmacist. “Dispensing” will be understood by the skilled addressee to mean that prescription information is used within a pharmacy setting to pick the medications on the prescription, write dosage or other instructions for a patient, and send the medication to the patient in preparation for taking.

Drugs may present risks when such drugs are contained in one or more known “drug types”. For example, venous thromboembolism drugs present a large threat due to increased risk of bleeding, particularly when co-administered. Each drug in the drug risk classification database may therefore be assigned a particular “drug type”, such as, for example, “controlled drug”, “non-formulary drug”, “venous thromboembolism (VTE) drug”, and “unscreened drug” among other possibilities which will be appreciated by the skilled addressee. This is the case in the example embodiment 10 shown, wherein the drug risk classification database further comprises a conditional associative data structure, arranged to assign a risk value (which may in turn be generated according to a supervised machine learning model and implemented using a machine learning module) to one or more conditions met by the first medication list. Such a data structure is shown, for example, in Table 3.

TABLE 3
Conditional drug type risk classifier

| Drug Data | Quantity | Risk Value |
|---|---|---|
| Controlled drugs prescribed | 1 or more drugs from | 5 |
| | Schedule 1 | |
| | 1 or more drugs from | 4 |
| | Schedule 2 | |
| | 1 or more drugs from | 3 |
| | Schedule 3 | |
| | 1 or more drugs from | 2 |
| | Schedule 4 | |
| | 1 or more drugs from | 1 |
| | Schedule 5 | |
| Non-formulary drugs prescribed | 1 or more | 2 |
| VTE drugs prescribed | None | 5 |
| | More than 1 | 5 |
| Unscreened drugs prescribed | 10 or over | 2 |
| | 5 to 9 | 1.5 |
| | 2 to 4 | 1 |
| | 1 | 0.5 |
| Previously prescribed | 1 or more | Equal to |
| medications not currently | | corresponding |
| prescribed | | Risk Value of |
| | | missed drug |

During step b) the present method compares the first list of medication (presently prescribed drugs) for the particular patient with the drug risk classification database to determine drug data comprising a series of risk values associated with the first list of medication. Using Patient 1 of Table 1 as an example, Patient 1 is prescribed Methadone which carries a risk factor of “5” according to Table 2. Methadone is a controlled drug in “Schedule 2” and thus carries an additional risk factor of “4”. This series of risk factors, forming the “drug data”, may be present in a data structure of any complexity, and in the present example is contained in a simple numerical array.

Embodiments may comprise patient data having a “second medication list” defining previously prescribed medications of a particular patient. The second medication list can be used, for example, to catch prescribing errors. Any medications from the second medication list which are not present in the first medication list may contribute to the calculation of the new risk value. Such contribution may be via a corresponding risk value attributed to the “potentially missed” medication. Such a risk value may be representative of the risk posed by said potential prescribing error, and may for example be equal to the risk value of the drug. Continuing with the present example, Patient 1 was previously prescribed Ciclosporin. During step b) of the present example method 10, the first medication list is also compared with the second medication list to satisfy elements of the conditional associative data structure shown in Table 3, and to populate the drug data with further risk values. As such, since Ciclosporin is no longer prescribed to Patient 1, the risk value of Ciclosporin, being “2”, is added to the drug data for Patient 1, as can be seen in Table 3 in combination with Table 2.

A drug risk classification data structure may further comprise information regarding known (or even theorized) drug-drug interactions, and the present method may therefore include a step (such as during step b)) of suitably allocating a risk value to the drug-drug interactions expected from the first medication list accordingly. In some embodiments, the drug-drug interaction information may be used or imported, by a system utilizing the present method from a remote source, which may be by way of a third party API. As an example, and using the present embodiment, each set of ten drug-drug interactions identified may carry an additional risk value of “1”, therefore ten drug-drug interactions would allocate a risk value of 1, twenty—a risk value of 2, thirty—a risk value of 3, and so on. Other embodiments may interpret drug-drug interactions by way of a modifier (such as a multiplier) of, for example, the known risk values of the one or more interacting drugs (which may in turn be generated according to a supervised machine learning model and implemented using a machine learning module). Any predetermined risk values, or methods of assignment of risk values, may be modifiable.

Step c) of the present example method 10 comprises the step of determining a new risk value using the drug data and the patient data. In this step of the present example method, the patient data is compared with a risk classification data structure to provide associated risk values according to the individual patient data. In this case, the number of days since the most recent medical appointment is compared with the risk classification data structure (an example can be seen in Table 4) in order to determine one or more individual risk values (which may in turn be generated according to a supervised machine learning model and implemented using a machine learning module) to be attributed to the patient data.

TABLE 4
Patient data risk classification data structure

| Patient | | Risk |
|---|---|---|
| Data | Period (days) | Value |
| Period since | 1 | 1 |
| last | 2 | 2 |
| appointment | 3 | 3 |

In the embodiment shown, the calculation of individual risk values to be attributed to the patient data is performed in step c). Embodiments will be appreciated wherein such a calculation is performed in step a), and upon retrieval of the patient data.

In the example shown, the patient data comprises only a value defining a period since a previous appointment. Additional embodiments will be appreciated wherein the patient data may also, or alternatively, comprise a value defining a period since a previous drug history review (DHx). Calculation of associated risk values (which may in turn be generated according to a supervised machine learning model and implemented using a machine learning module) according to such a period may use a data structure such as that shown in Table 5.

TABLE 5
Period since a drug history review

| | Period | |
|---|---|---|
| Patient Data | (days) | Risk Value |
| Period since | 1 | 1 |
| last drug | 2 | 2 |
| history review | 3 | 3 |
| (DHx) | 4 | 4 |

Such individual risk values, like for the drug data, may be contained within any suitable data structure, such as, for example, a numerical array.

Further in step c), risk values associated with the patient data and the drug data are summed to provide a new risk value for the particular patient. Other examples will be appreciated wherein the risk values associated with the patient data and the drug data are processed by a machine learning module to provide a new risk value for the particular patient. Steps d) and e) of the method 10 comprise changing the current risk value to the new risk value and inserting the patient data into a patient database entry according to the unique identifier, which in the present embodiment may comprise overwriting an existing database entry stored for the particular patient. Embodiments will be appreciated wherein only the current risk value is overwritten to the database entry according to the unique identifier.

Step f) of the present embodiment comprises ordering the unique identifiers according to their corresponding risk values.

Embodiments will be appreciated wherein the patient data comprises one or more clinical observations, wherein the one or more clinical observations may present evidence of reduced kidney or liver functionality, or a particular drug allergy. Such clinical observations may be used in the present method to identify contraindications or increase risk values of particular risk factors (for examples drugs) accordingly. For example, if a patient has a known allergy to amoxicillin or other penicillin derivatives, any penicillin derivative may receive a higher than normal risk value. Such an effect of an allergy or other contraindication may present as a risk value modifier (such as a multiplier) for example. In the example shown in Table 1, the patient data comprises clinical observation data detailing disease information which may cause the present method to allocate a risk value to drugs contraindicated for said disease information.

TABLE 6
Clinical observations risk classification data structure

| Clinical Observation | Risk Value |
|---|---|
| Medication allergies and | 5 |
| prescribed | |
| Missed doses of drugs | Equal to corresponding |
| | Risk Value of missed |
| | drug |
| Each alert requiring pharmacist | 5 |
| action | |
| Any blood result out of healthy | 2 |
| range | |
| Any AKI stage | 2 |

The present method 10 will apply for a pharmacist ward page, a tech ward page and a group ward page (for example, a weekend list displaying all patients from all wards in a group).

A system implementing the present method may permit access to, or viewing of, different information by a user according to the specific role of said user. In such a system, separate role-specific applications may contain data or information relating to a specific role—for example, a pharmacist may be permitted access to pharmacist-specific information such as drug data and clinical observation data, among others which will be appreciated. Similarly, a technician or a nurse may be permitted access to corresponding role-specific information. The system may allocate a specific role to information in order to permit access to said information only by individual performing the allocated role(s). Such roles may be discerned by the system using, for example, user log-in information. The system may also allocate certain information within the patient data or drug data, such as unique identification information for example, as generic and viewable by all roles. In such a system the present method may further comprise a step of determining a role of a user, and an additional step of displaying role-specific data to said user, wherein said role specific data may be predetermined as described above.

If a patient classifier, such as a location/ward, consultant or team, comprises a set list of patients, patients within said classifier may be grouped into classifier-specific unique identifier lists such that said patients are prioritized within their associated group. Ordered unique identifier lists can then, in such preferable examples of the present invention be viewed in their respective groups such as for each consultant, team, or location/ward.

It is possible to identify, using the present method, two or more patients in a patient group having the same current risk value, and as such in the ordering step f) it may be unclear the order in which each of these patients should be seen according to the corresponding at-risk status.

In such a case, the present method preferably provides one or a number of check steps wherein duplicate risk values are identified and a more detailed ordering is performed. The more detailed ordering may require an additional analysis of, for example, the drug data produced for each patient such that a comparison may be performed. Therefore, in systems carrying out re-ordering of duplicate risk values, drug data corresponding to each patient is therefore stored in the database along with the patient data. The drug data for each patient may therefore be accessed in the event of a duplicate risk value such that a comparison may be performed between the drug data of a first patient having a risk value and the drug data of a second patient having a second risk value equal to the first risk value.

Such a comparison may, for example, identify the patient or patients prescribed with a greater number of drugs in “higher” risk categories. Patients may then be ordered according to the corresponding number of drugs in each risk category, descending in severity of risk. For example, if three patients are identified to have the same risk value following step 0 of the present method, each of the three patient's unique identifiers are then ordered according the number of prescribed drugs (those in the first medication list) appearing in “Schedule 1”. Should this result in one or more patient still having the same risk value, each of these one or more patient's unique identifiers are then ordered according the number of prescribed drugs appearing in “Schedule 2”, then “Schedule 3” and so on until there are no further reordering steps which can be performed or until each patient's unique identifier appears at a different place in the order—whichever occurs first. Additional reordering may be performed according to, for example, “number of drug-drug interactions”, or one or more “clinical observations” which may include blood markers indicating kidney competence or liver competence.

An example will now be described with respect to Table 7 and Table 8.

TABLE 7
Example

| | | | | | Risk | | | | |
|---|---|---|---|---|---|---|---|---|---|
| | High | Medium | Caution | | for | | Risk | | |
| | Risk | Risk | Risk | Days | days | | for | | New |
| Patient | Drugs | Drugs | Drugs | without | without | UV | UV | | Risk |
| Identifier | Total | Total | Total | DHx | DHx | Drugs | Drugs | . . . | Value |
| Pat | 7 | 3 | 0 | 2 | 2 | 3 | 1 | . . . | 13 |
| Jil | 0 | 2 | 0 | 1 | 1 | 2 | 1 | . . . | 4 |
| Tim | 3 | 0 | 1 | 4 | 4 | 0 | 0 | . . . | 8 |
| Kat | 2 | 0 | 0 | 5 | 4 | 1 | 0.5 | . . . | 6.5 |
| Elis | 6 | 0 | 0 | 0 | 0 | 1 | 0.5 | . . . | 6.5 |

In Table 7, the column labelled “ . . . ” represents any number of additional columns of patient data or drug data which may be present in such a table or database entry for consideration during one of the steps of the present method, such as step c) or a reordering step during or following step f). Such additional patient data or drug data may comprise any weighting factors, biasing factors or activation factors generated by or obtained from a machine learning module such as that described herein. Such weighting factors, biasing factors or activation factors may be derived from training data in a supervised neural network generated by the machine learning module. Example such input data (such as patient data, drug data and example weighting/biasing factors) is provided below in Table 10, Table 11 and Table 12.

In Table 7, risk values determined during step c) are shown in the column “New Risk Value”. The values listed in the “New Risk Value” column are determined, in the example shown, from the sum of values in the corresponding row relating to “High Risk Drugs Total”, “Medium Risk Drugs Total”, “Caution Risk Drugs Total”, “Risk for days without DHx”, and “Risk for UV Drugs” present in each corresponding row. These values contributing to the sum “New Risk Value” are provided in bold in Table. 7 for clarity. As can be seen, the corresponding risk values for the risk classifiers “Days without DHx” and “UV Drugs” do not always equal the values assigned to these classifiers. These risk values for each risk classifier are, in the example shown, calculated using a corresponding algorithm assigning an appropriate risk value according to the risk presented by the risk classifier value. Embodiments will be appreciated wherein the risk value assigned to one or more risk classifier value is equal to the risk classifier value. In the embodiment shown, the “New Risk Value” is determined as a sum. Embodiments will be appreciated wherein the “New Risk Value” is determined according to any suitable formula or algorithm. In the “New Risk Value” column of the example shown in Table 7, it can be seen that Kat and Elis have been assigned the same risk value and will therefore appear at the same place in the ordered unique identifiers following step f).

In this example, if any patient has the same risk value as one or more other patients, one or more reordering steps are performed until the patients comprise different risk values to one another. In the example, the reordering steps consider the individual risk values presented by each risk factor in-turn, so using Table 7, individual risk factors from “High Risk Drugs Total” to “Risk for UV Drugs” will be considered in turn until a suitable reordering is achieved. Using Table 7, it can be seen that Elis would appear higher than Kat in the reordered unique identifiers since Elis is prescribed six “High Risk Drugs” whereas Kat is only prescribed two “High Risk Drugs”. The reordered unique identifiers would therefore be as shown in Table 8.

TABLE 8
Reordered unique identifiers following Example
Patient Identifier
Pat
Tim
Elis
Kat
Jil

In optional embodiments, a system may be arranged to only accept contributions or amendments to risk values from appropriate or authorized personnel. For example, a technician may confirm that a patient has been “seen today”, but a system may only permit modification of the “period since a previous appointment” or “period since a previous drug history review” upon confirmation from an approved medical professional, such as a doctor or a pharmacist. Other embodiments will be appreciated wherein any other contributions or modifications to any other risk factors or risk values may be controlled by permissions or authorizations.

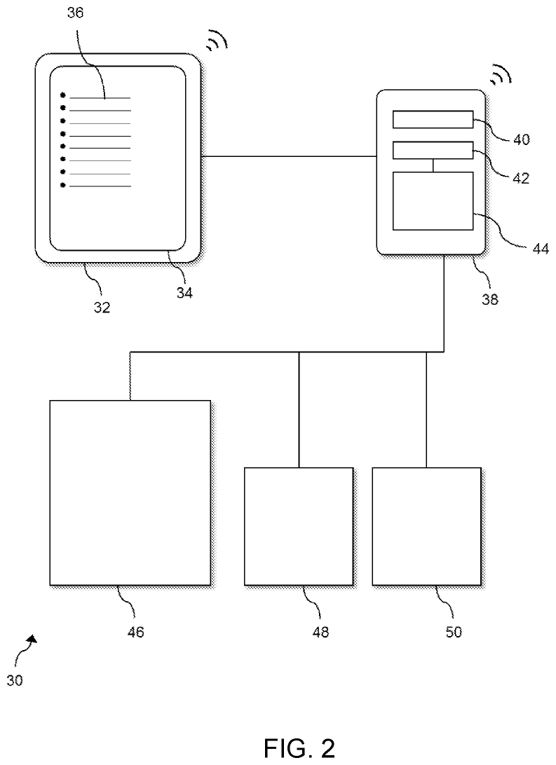

FIG. 2 shows an example embodiment 30 of a risk stratification system according to the third aspect of the present invention arranged to perform the method of the first aspect of the present invention, and arranged to perform the example methods described above and shown in FIG. 1.

The system 30 comprises a first processing device 32 having a display output 34 in the form of a touch sensitive screen 34. The first processing device 32 in the example shown takes the form of a hand-held tablet computer 32. Embodiments will be appreciated wherein the first processing device 32 is any suitable processing device, and may optionally be a desktop computer. The touch-sensitive screen 34 is preferably arranged to accept input from a user by touching responsive portions of the touch-sensitive screen 34. The screen 34 is arranged to display an ordered list of unique identifiers 36 to a user. In the embodiment shown, the user (not shown) is a pharmacist.

The system 30 further comprises a second processing device 38 having a processor 40 arranged to process a computer program product arranged to perform the method steps of first aspect of the present invention. The second processing device 38 in the example shown takes the form of a sever 38 in digital communication with the tablet computer 32, wherein the digital communication is a wireless communication. Embodiments will be appreciated wherein the digital communication is a wired communication, and additional embodiments will be appreciated wherein there is only one processing device arranged to perform the functions of both of the first processing device 32 and the second processing device 38 of the example embodiment described.

The server 38 comprises a memory 42 having stored thereon a patient database 44 comprising patient data and drug data, each assigned to individual patient entries on the patient database 44 according to a patient unique identifier. The server 38 is in further digital communication with a drug risk classification database 46, which is interpreted by the processor 40 of the server 38 using a third party API (not shown).

The server 38 is in further digital communication with a procurement management system 48, the procurement management system 48 arranged to receive automated procurement requests from the server 38.

The server 38 is in further digital communication with a clinical observation database 50, the clinical observation database 50 having a store of, for example, medical records which may be updated by any number of local or national medical departments (not shown) following clinical observations of a particular patient. Preferably the unique identifiers 36 utilised by the server 38 correspond to unique identifiers utilised by the clinical observation database 50 such that observations obtained for a particular patient can be sourced by the server 38 and recorded on the correct patient entry of the patient database 44.

In use, a pharmacist (not shown) carries the tablet computer 32 on a ward round for individual patient visitation, said visitation being performed in a patient order 36 determined by the server 38 using the method according to the first aspect of the present invention and substantially as hereinbefore described. The server 38 communicates the ordered or reordered unique identifiers to the tablet computer 32 for display to the pharmacist on the screen 34. The pharmacist may interact with to screen 34 to modify, add or remove patient data or drug data from the patient database 44 contained within the memory 44 of the server 38. Thereby, the pharmacist can determine live updates to the patient database 44 via the tablet computer 32, and the server 38 may correspondingly communicate live updates to the ordered unique identifiers 36 to the tablet computer 32. Updated information received from the drug risk classification database 46 may also contribute to the reordering of the patient unique identifiers 36 by the server 38. Further live updates to the ordered unique identifiers 36 by the server 38 may be driven by information received from the clinical observation database 50, which may, for example, include a recent blood test result indicating compromised kidney functionality. Such a blood test result may cause an additional risk value to be attributed to a particular patient within the ordered unique identifiers 36 such that said patient appears higher on the list 36 displayed to the pharmacist.

A first medication list (not shown) of a particular patient appearing in the ordered list 36 may be approved by a pharmacist following a visitation. Followed authorised approval, drugs in said first medication list may change in status from “unscreened” to “screened” and may therefore confer a lower risk value upon said patient's new risk value, and therefore may cause a patient to appear lower on the ordered list 36. The pharmacist, following a visitation, may activate a “seen today” feature of the screen, such that a new risk value is assigned to the visited patient, wherein that risk value is zero. Such a “seen today” feature in the embodiment shown carries a time period for which the reassigned risk value is maintained. At midnight each day, the method of the first aspect is performed and the new risk value represents an updated risk value, which may be higher than zero.

In the embodiments described, the example drug classification database (Table 2) comprises drug entries having a drug identifier and a corresponding drug risk value. In embodiments, allocation of the drug risk values to the corresponding drug identifiers may be performed according to drug classifiers. Examples of such drug classifiers are shown in Table 9, comprising a series of questions answered by an authorised professional. In other embodiments, such answers or other suitable classifiers may be populated or calculated automatically by an algorithm, and may use third party databases, tools and/or information to inform said populating/calculating.

TABLE 9
Example drug risk classification tool

| Drug Name | | Methadone |
|---|---|---|
| Drug Level Monitoring | | Yes |
| Nephrotoxic | | Yes |
| Loss of symptom | Missed-dose risk | Yes |
| control | | |
| Either/Or | Time-critical drug | Yes |
| or | Delay non-critical | |
| Neither | but affects future | No |
| (not both) | management | |
| Electrolyte/blood/other monitoring | | No |
| needed | | |
| Daily drug review required | | No |
| Side effects/harm | Detrimental if | Yes |
| to patient | prescribed | |
| | incorrectly | |

| | |
|---|---|
| Drug risk value | 5 |

In the example shown in Table 9, an example drug risk classification tool is shown arranged to provide a drug risk value to, in the example shown, methadone. Additional embodiments will be appreciated wherein the drug risk classification tool utilizes any suitable drug risk classifier, examples of which are described herein.

As a further processing step which may be useful for incorporation into step c) of the example embodiments discussed, there is provided a machine learning module used to generate a supervised model of weighting, biasing and activation factors used in the calculation of a new risk value. Such weighting, biasing and activation factors may, for example, be useful for inclusion into the column labelled “ . . . ” in Table 7

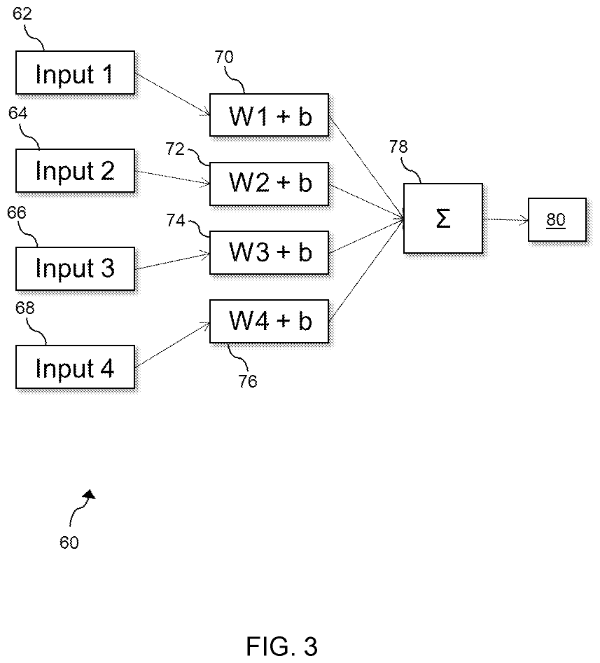

Using such a machine learning module, the one or more computing devices may allocate the associated input patient data and/or drug data with the appropriate weighting and biasing factors. An example workflow 60 is depicted in FIG. 3 in which the resulting generating of a new risk value 80 may therefore include a cumulative sum 78 of individually-weighted (W1, W2, W3, W4) and (optionally collectively) biased (b) input data values 62, 64, 66, 68 performed according to the supervised model. For example Input 1 (age) 62 may have the associated weighting factor W1 (from Table 10 below) applied thereto 70. Similarly, Input 2 (admissions in the last 12 months) 64, Input 3 (current length of stay) 66, and Input 4 (number of high-risk medications prescribed) 68 may be correspondingly weighted 72, 74, 76 (W2, W3, W4) according to Table 10 below. The weighted Inputs 1 to 4 may then be collectively biased according to a global, patient-specific biasing factor (b, such as according to historic patient data), and cumulatively summed (Σ) to achieve a (weighted sum) activation factor 78 used in generating a new risk value 80 (which may be equal to the activation factor).

Successive training and retraining of the supervised model of the machine learning module may be undertaken as required. For example, the weighting factors provided in Tables 10 to 12 below may be adjusted based on future model training, which may follow a protocol as follows:
load data;
build training pipeline;
evaluate the quality of the model;
train the model;
save the model; and
perform the method according to the present invention utilising the model in generation of the new risk value, for example, at step c).

TABLE 10
Example weighting factors from machine learning module
supervised model

| Input patient data/drug data | Weighting |
|---|---|
| Age of patient | >/= 75 = 0.6, <75 = 0.3 |
| Number of admissions in the last 12 | 0.2 |
| months | |
| Current length of stay | >4 days = 0.9, >3 = 0.7, |
| | >2 = 0.4>, 1 >= 0.1 |
| Number of high risk medications | 0.9 |
| prescribed | |
| Number of medium risk medications | 0.5 |
| prescribed | |
| Total number of medications prescribed | >/=5 = 0.4, <5 = 0.2 |
| Weight of patient | 0.2 |
| Previous occurrence of patient data | 0.5 |
| Number of unscreened medications | 0 to 3 = 0.2, 4 to 8 = 0.4, >9 = 0.8 |

TABLE 11
Example weighting factors from machine learning module
supervised model

| Input patient data/drug data | Weighting |
|---|---|
| Pharmacist follow up in x number of days | 0.6 |
| DHx status | R = 0.9, O = 0.6, C = 0 |
| Number of pharmacist-alerted | 0.7 |
| medications | |
| Number of medications missed present | 0 to 2 = 0.2, 3 to 4 = 0.4, >4 = 0.6 |
| on first medications list but missing from | |
| second medications list | |
| Number of medication rules not followed | 0.8 |
| Number of medication rules requiring | 0.8 |
| review | |
| Medication allergy and corresponding | 0.8 |
| medication prescribed | |
| VTE medication not prescribed | 0.9 |
| VTE medication duplicated | 0.9 |
| Any stage AKI | 0.7 |

TABLE 12
Example weighting factors from machine learning module
supervised model

| Input patient data/drug data | Weighting |
|---|---|
| Number of non-formulary drugs | 0.4 |
| Number of blood results | 0.5 |
| exceeding/subsceeding healthy threshold | |
| Number of predicted drug-drug | 0.4 |
| interactions | |
| Number of days without a drug history | 0.5 |
| Number of medications which require | 0.1 |
| counselling-pre-defined | |
| Number of medications which require | 0.1 |
| counselling-free type | |
| Number of controlled drugs prescribed | 0.1 |
| Controlled drug schedule | 0.1 |
| High risk medications not prescribed in | 0.9 |
| first medication list compared with | |
| second medication list | |
| Number of caution medications | 0.4 |
| prescribed | |

It will be appreciated that the above described embodiments are given by way of example only and that various modifications may be made to the described embodiments without departing from the scope of the invention as defined in the appended claims. For example, in the embodiments shown and described, the patient data comprises a current risk value and the method involves calculating a new risk value and changing the current risk value to the new risk value. Embodiments will be appreciated wherein allocation of a new risk value according to a unique identifier may occur in a transient memory and thus there may be no need for a “current risk value” in the patient data. Therefore, the “storage” step d) may involve merely the storage within transient memory only. Additionally, in the embodiments shown and described, a specific range of values are used to represent the “risk values”. In practice, these risk values may take the form of any value, either integer, floating point number, binary or other value appreciable by the skilled addressee. In the embodiments described, the unique identifiers are a patient name. Embodiments will be appreciated wherein the unique identifier may be anonymized or encrypted at any point in the method or system, for example during transmission from one processing device to another. The first processing device may comprise a decryption key, for example, to decrypt encrypted unique identifiers for viewing by authorized personnel only. A system performing the present method may carry out audit tracking in order to ensure end-to-end evidence of compliant use of medication. When an audit, of medication or dose regimen for example, is being carried out, a patient may receive an alert if the required criteria for the audit is achieved. A system user may action the audit result and store any relevant data from said audit. A system implementing the present method may comprise a chatbot arranged to link collected data and present the data to a system user when required.

In some examples, the present system preferably allows you to track specific patients that have been identified by individual users/personnel displayed on a dashboard displaying the ordered unique identifiers. Such a dashboard may also display customizable alerts such as a display of the number of new patients, those requiring medicine reconciliation, those needing an immediate review (such as “today”), and provide an alert for specific medications.

Personnel may, in preferable embodiments, delete any data held in a database to ensure local data protection legislation compliance.

It will also be appreciated that embodiments of the present invention may include the step of: overriding a reordering of the unique identifier list or generated new risk value should an individual or personnel identify an error. As such, in systems implementing the present invention, all components are preferably user-interactive and provide updates in real time. Any alerts may be customized in order for them to be displayed if applicable to the patient. Alerts may be interactive to provide further information or indicate completion of a specific task.

Having thus described the aforementioned invention, what is claimed is:

1. A method of data stratification for management of a patient group according to individual risk factors, the method comprising the steps of:
a) by one or more computing devices, receiving a patient data, the patient data comprising:
i. a unique identifier;
ii. a first medication list comprising currently prescribed medications;
b) by one or more computing devices, producing a drug data by comparing the first medication list with a drug risk classification database;
c) by one or more computing devices, determining a new risk value using the drug data and the patient data;
d) by one or more computing devices, storing the patient data and the new risk value, and optionally the drug data, into a memory; and
e) by one or more computing devices, accessing the memory and ordering the unique identifiers into ordered unique identifiers, the ordered unique identifiers being ordered according to the corresponding new risk values.

2. A method as claimed in claim 1, wherein the patient data further comprises:
iii. a current risk value; and
wherein step c) further comprises, by the one or more computing devices, changing the current risk value to the new risk value.

3. A method as claimed in claim 2, wherein the patient data further comprises:
iv. a period since a previous appointment.

4. A method as claimed in claim 3, wherein the method further comprises the step of:
f) by one or more computing devices, generating a report of the ordered unique identifiers.

5. A method as claimed in claim 3, wherein step c) comprises, by the one or more computing devices:
determining that the period since a previous appointment is zero; and
determining the new risk value of zero.

6. A method as claimed in claim 5, wherein the patient data further comprises:
v. a period since a previous drug history review.

7. A method as claimed in claim 6, wherein step c) comprises, by the one or more computing devices:
determining that the period since a previous drug history review is zero; and
determining the new risk value of zero.

8. A method as claimed in claim 7, wherein the patient data further comprises:
vi. a second medication list comprising previously prescribed medications.

9. A method as claimed in claim 8, wherein the producing of drug data of step b) further comprises, by the one or more computing devices:
comparing the second medication list with the first medication list.

10. A method as claimed in claim 9, wherein the patient data further comprises:
vii. a clinical observation data;

11. A method as claimed in claim 10, wherein the clinical observation data comprises one or more selected from the group: an allergy classifier; a missed dose classifier; a renal competency classifier; a hepatic competency classifier; a blood marker classifier; a patient demographic classifier; an age classifier; a race classifier; a genetic marker classifier; a disease classifier; a comorbidity classifier.

12. A method as claimed in claim 11, wherein the drug risk classification database comprises one or more drug entries contained in one or more drug category entries, wherein each drug entry comprises a corresponding drug risk value.

13. A method as claimed in claim 12, wherein the drug category entries comprise one or more selected from the group:
i. unverified drugs;
ii. controlled drugs;
iii. non-formulary drugs;
iv. venous thromboembolism drugs;
v. drug-drug interactions.

14. A method as claimed in claim 13, wherein the drug data comprises at least one drug category quantity, wherein the at least one drug category quantity corresponds to a number of the drug entries within said drug category which are present in the first medication list.

15. A non-transitory storage media comprising software code portions operable when executed to perform the method steps of:
a) by one or more computing devices, receiving a patient data, the patient data comprising:
i. a unique identifier;
ii. a first medication list comprising currently prescribed medications;
b) by one or more computing devices, producing a drug data by comparing the first medication list with a drug risk classification database;
c) by one or more computing devices, determining a new risk value using the drug data and the patient data;
d) by one or more computing devices, storing the patient data and the new risk value, and optionally the drug data, into a memory; and
e) by one or more computing devices, accessing the memory and ordering the unique identifiers into ordered unique identifiers, the ordered unique identifiers being ordered according to the corresponding new risk values.

16. The non-transitory storage media according to claim 15, wherein the non-transitory storage media comprises a computer-readable medium on which the software code portions are stored, wherein the media is directly loadable into an internal memory of the processing device.

17. A risk stratification system arranged to perform the method steps of:
a) by one or more computing devices, receiving a patient data, the patient data comprising:
i. a unique identifier;
ii. a first medication list comprising currently prescribed medications;
b) by one or more computing devices, producing a drug data by comparing the first medication list with a drug risk classification database;
c) by one or more computing devices, determining a new risk value using the drug data and the patient data;
d) by one or more computing devices, storing the patient data and the new risk value, and optionally the drug data, into a memory; and
e) by one or more computing devices, accessing the memory and ordering the unique identifiers into ordered unique identifiers, the ordered unique identifiers being ordered according to the corresponding new risk values;
the system comprising a processing device having a processor arranged to process software code portions of a non-transitory storage media comprising software code portions operable when executed to perform steps a) through e).
